(19) United States
(12) Patent Application Publication
(10) Pub. No.: US 2011/0152942 A1
(43) Pub. Date: Jun. 23, 2011

(54) Method and Apparatus for Using a Multi-Hinged Longitudinal Member

(75) Inventors: YoungHoon Oh, Montville, NJ (US); John W. McClellan, III, Omaha, NE (US); Mahmoud F. Abdelgany, Rockaway, NJ (US)

(73) Assignee: CUSTOM SPINE, INC., Parsippany, NJ (US)

(21) Appl. No.: 12/641,241

(22) Filed: Dec. 17, 2009

(51) Int. Cl. A61B 17/88; A61B 17/56

(52) U.S. Cl. 606/279; 606/86 A

(57) ABSTRACT

A method and apparatus for using a multi-hinged longitudinal member including providing the multi-hinged longitudinal member, including a primary first hinge component pair; and an auxiliary first hinge component pair; providing a primary control device including: a primary control arm pair; and a primary second hinge component pair; providing an auxiliary control device, including an auxiliary second hinge component pair; and an auxiliary control arm pair; creating a primary hinge by connecting each primary first hinge component to a unique primary second hinge component to create a primary hinge; creating a auxiliary hinge by connecting each the auxiliary first hinge component to a unique auxiliary second hinge component to create an auxiliary hinge; rotating the multi-hinged longitudinal member along different axes through manipulation of at least one of the primary control device and the auxiliary control device; and setting the multi-hinged longitudinal member in the desired position.

### BACKGROUND

1. Technical Field

The embodiments described herein generally relate to medical devices, and, more particularly, to medical devices used for spinal implant surgeries.

2. Description of the Related Art

Traditional surgical procedures for pathologies located within the body can cause significant trauma to the intervening tissues. These procedures often require a long incision, extensive muscle stripping, prolonged retraction of tissues, denervation and devascularization of tissue. These procedures can require operating room time of several hours and several weeks of post-operative recovery time due to the destruction of tissue during the surgical procedure. In some cases, these invasive procedures lead to permanent scarring and pain that can be more severe than the pain leading to the surgical intervention.

The development of percutaneous surgical procedures has yielded a major improvement in reducing recovery time and post-operative pain because minimal dissection of tissue, such as muscle tissue, is required. For example, minimally invasive surgical techniques are desirable for spinal and neurosurgical applications because of the need for access to locations within the body and the danger of damage to vital intervening tissues. While developments in minimally invasive surgery are steps in the right direction, there remains a need for further development in minimally invasive surgical instruments and methods. For example, conventional surgical instruments used during minimally invasive surgical procedures provide limited movement surgery and offer limited depth control during the procedure. These shortcomings to convention minimally invasive surgical instruments frequently raise the risk of additional morbidity to a patient undergoing a minimally invasive surgical procedure.

### SUMMARY

In view of the foregoing, an embodiment herein provides a method of setting a multi-hinged longitudinal member in a desired position during a spinal surgery, the method comprising providing the multi-hinged longitudinal member, wherein the multi-hinged longitudinal member comprises: a primary first hinge component pair; and an auxiliary first hinge component pair; providing a primary control device, wherein the primary control arm comprises a primary control arm pair; and a primary second hinge component pair, wherein each primary control arm of the primary control arm pair is coupled to a primary second hinge component of the primary second hinge component pair; providing an auxiliary control device, wherein the auxiliary control arm comprises: an auxiliary second hinge component pair; and an auxiliary control arm pair, wherein each auxiliary control arm of the auxiliary control arm pair is coupled to an auxiliary second hinge component of the auxiliary second hinge component pair; creating a primary hinge by connecting each primary first hinge component to a unique primary second hinge component to create a primary hinge; creating a auxiliary hinge by connecting each the auxiliary first hinge component to a unique auxiliary second hinge component to create an auxiliary hinge; rotating the multi-hinged longitudinal member along different axes through manipulation of at least one of the primary control device and the auxiliary control device; and setting the multi-hinged longitudinal member in the desired position.

Such a method may further comprise setting a center hinge on the multi-hinged longitudinal member; holding the primary hinge stationary; and articulating the auxiliary hinge. Moreover, such a method may further comprise rotating the multi-hinged longitudinal member along a lateral axis of the multi-hinged longitudinal member; articulating the primary hinge in a first direction; and articulating the auxiliary hinge in a second direction, wherein the first direction is a complement of the second direction. In addition, such a method may further comprise: rotating the multi-hinged longitudinal member along a longitudinal axis of the multi-hinged longitudinal member; articulating the primary hinge in a first direction; and articulating the auxiliary hinge in the first direction.

Another embodiment herein provides a method of setting a multi-hinged longitudinal member in a desired spatial relationship with spinal elements during minimally invasive spinal surgery, the method comprising providing a longitudinal member comprising multiple hinge components; providing an inserter device comprising means for actuating the multiple hinge components; and actuating the multiple hinge components of the longitudinal member.

In such a method, the actuation of the multiple hinge components may allow for rotation of the longitudinal member along different axes of rotation. Moreover, the longitudinal member may comprise elongated side surfaces, wherein the multiple hinge components are configured along the side surfaces. Furthermore, the longitudinal member may comprise a cylindrical body. In addition, the multiple hinge components may comprise dimples indented into the longitudinal member. Additionally, the multiple hinge components may comprise projections outwardly protruding from the longitudinal member.

Moreover, in such a method, the means for actuating the multiple hinge components may comprise multiple projections that insert into the dimples. In addition, the means for actuating the multiple hinge components may comprise multiple notches that engage the projections. Furthermore, such a method may further comprise providing multiple inserter devices comprising means for actuating the multiple hinge components.

Another embodiment herein provides an apparatus for setting a multi-hinged longitudinal member in a desired spatial relationship with spinal elements during minimally invasive spinal surgery, the apparatus comprising a longitudinal member comprising multiple hinge components; and at least one inserter device comprising means for actuating the multiple hinge components, wherein the actuation of the multiple hinge components allows for rotation of the longitudinal member along different axes of rotation.

In such an apparatus, the longitudinal member may comprise elongated side surfaces, wherein the multiple hinge components are configured along the side surfaces. Moreover, the longitudinal member may comprise a cylindrical body. Furthermore, the multiple hinge components may comprise dimples indented into the longitudinal member. In addition, the multiple hinge components may comprise projections outwardly protruding from the longitudinal member. Additionally, the means for actuating the multiple hinge components may comprise multiple projections that insert into the dimples. In addition, the means for actuating the multiple hinge components may comprise notches that engage the projections.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIGS. 1(A) through 1(C) illustrate a schematic diagram of a multi-hinged longitudinal member according to a first embodiment herein;

FIG. 2 illustrates a schematic diagram of a hinge configuration of a multi-hinged longitudinal member according to a second embodiment herein;

FIG. 3 illustrates a schematic diagram of a hinge configuration of a multi-hinged longitudinal member according to a third embodiment herein;

FIG. 4 illustrates a schematic diagram of a hinge configuration of a multi-hinged longitudinal member according to a fourth embodiment herein;

FIG. 5 illustrates a schematic diagram of a hinge configuration of a multi-hinged longitudinal member according to a fifth embodiment herein;

FIGS. 6(A) through 6(C) illustrate a schematic diagram of a multi-hinged longitudinal member with an unsecured insertion device according to an embodiment herein;

FIGS. 7(A) through 7(C) illustrate a schematic diagram of a multi-hinged longitudinal member with a secured insertion device according to an embodiment herein;

FIGS. 8(A) through 8(C) illustrate a schematic diagram of a left inner shaft of the insertion device according to an embodiment herein;

FIGS. 9(A) through 9(C) illustrate a schematic diagram of a right inner shaft of the insertion device according to an embodiment herein;

FIGS. 10(A) through 10(C) illustrate a schematic diagram of an outer sleeve of the insertion device according to an embodiment herein;

FIGS. 11(A) through 11(C) illustrate a schematic diagram of a rotating knob of the insertion device according to an embodiment herein;

FIGS. 12(A) through 12(D) illustrate a schematic diagram of an actuator of the insertion device according to an embodiment herein;

FIGS. 12(E) through 12(G) illustrate a schematic diagram of another actuator of the insertion device according to an embodiment herein;

FIGS. 13(A) through 13(D) illustrate a schematic diagram of a base connector of the insertion device according to an embodiment herein;

FIGS. 14(A) through 14(C) illustrate schematic diagram of a hinge of the insertion device according to an embodiment herein;

FIGS. 15(A) through 15(B) illustrate a schematic diagram of a pin of the insertion device according to an embodiment herein;

FIG. 16 illustrates a schematic diagram of another insertion device according to an embodiment herein;

FIGS. 17(A) and 17(B) illustrate a schematic diagram of yet another insertion device according to an embodiment herein; and

FIGS. 18 and 19 are flow diagrams illustrating methods according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

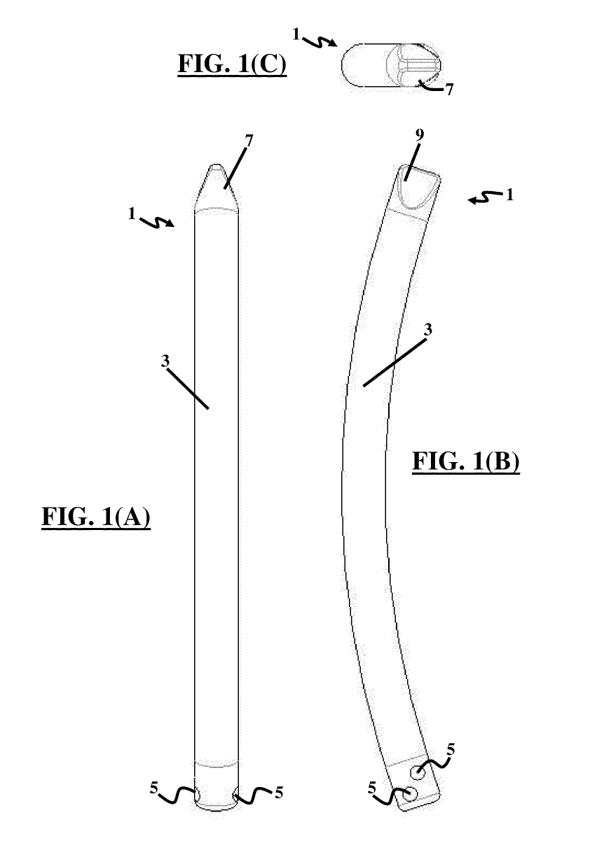

As mentioned above, there remains a need for a novel implant and method for use during minimally invasive surgical procedures (e.g., spinal surgeries that utilize a small incision) that allows greater manipulation of the surgical instrument (e.g. during placement of a surgical implant, such as a spinal rod) during surgery and allows greater depth control. The embodiments herein provide a multi-hinge longitudinal member that may function with an insertion device or a minimal access/invasive insertion device for spinal surgery, and more specifically a multi-hinged longitudinal member that holds and locks in a vertical or semi-vertical position from one end and allows rotation along multiple axes within an incision. Referring now to the drawings, and more particularly to FIGS. 1(A) through 19, there are shown preferred embodiments.

FIGS. 1(A) through 5 illustrate various embodiments of a longitudinal member 1 according to the embodiments herein. In each of the embodiments shown in FIGS. 1(A) through 5, the hinge components 5 may be embodied as dimples indented into the longitudinal member 1 or as projections outwardly protruding from the longitudinal member 1.

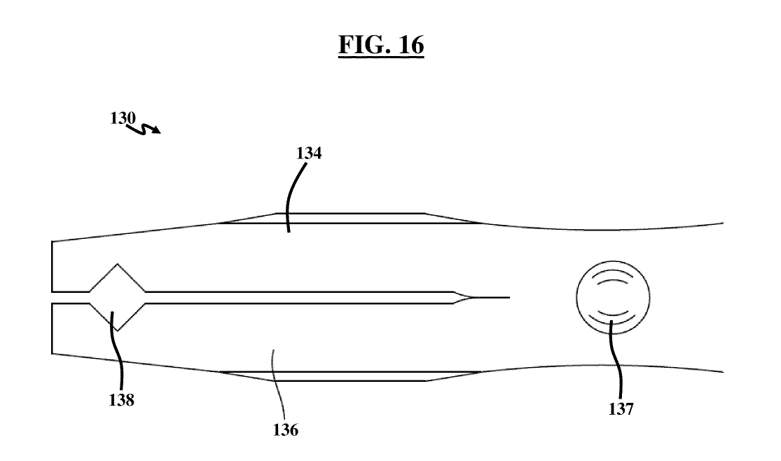

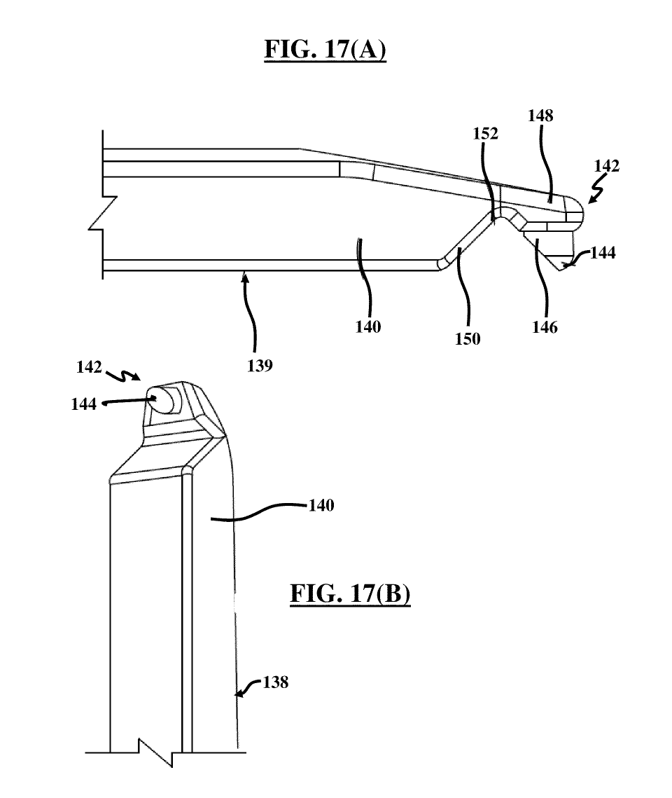

FIGS. 1(A) and 1(C) illustrate a schematic diagram of a multi-hinged longitudinal member 1 according to a first embodiment herein. As shown, multi-hinged longitudinal member 1 includes a main body 3 with a plurality of first hinge components (or pivot couplings) 5 cut therein. In addition, longitudinal member 1 may include an elongated member 7 and a connecting cavity 9. The plurality of first hinge components 5 are configured to mate with an insertion device (e.g., nubs 14, 34 on gripping arms 12, 32, as shown in 8(A) through 9(C), or with notch 138, or tip 144 shown in FIGS. 16 through 17(B) and discussed in further detail below). In this regard, nubs 14, 34 can grip the hinge components 5 of the longitudinal member 1 especially when the first hinge components 5 are embodied as dimples indented in the longitudinal member 1. In addition, elongated member 7 may mate with connecting cavity 9 on a second end of a second longitudinal member 1 to thereby chain more that one longitudinal member 1 together. Although multi-hinged longitudinal member 1 may be configured as a spinal rod, as shown in FIG. 1(A); multi-hinged longitudinal member 1 is not limited to a spinal rod and may include any surgical implant and have any suitable configuration.

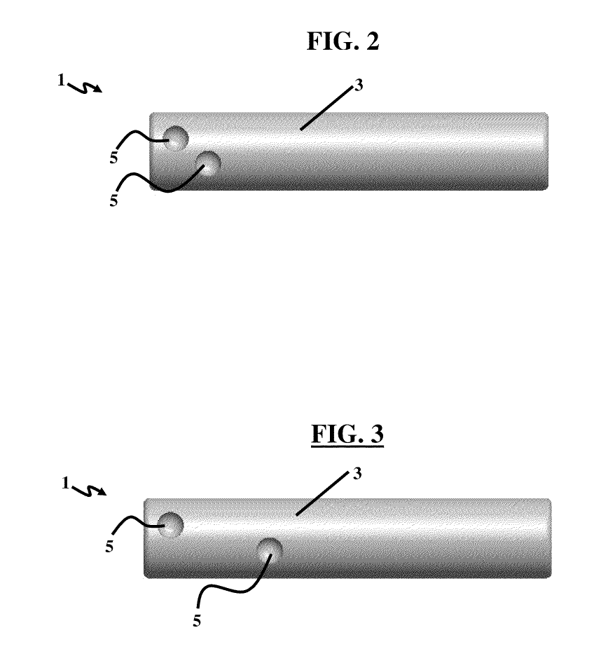

FIG. 2, with reference to FIGS. 1(A) through 1(C), illustrates a schematic diagram of a configuration of hinge components 5 on a multi-hinged longitudinal member 1, according to a second embodiment herein. The hinge configuration shown in FIG. 2 has two pairs of first hinge components 5 closely clustered at one end of multi-hinged longitudinal member 1 on main body 3. While only one first hinge component 5 of the pair is show, the other first hinge component 5 of the pair that is not shown is evenly spaced on the opposite longitudinal side of main body 3. In addition, each pair of first hinge components 5 is offset from the other pair; for example, one pair of first hinge components 5 is set higher than the other pair of first hinge components 5. FIG. 3, with reference to FIGS. 1(A) through 2, illustrates a schematic diagram of a configuration of hinge components 5 on a multi-hinged longitudinal member 1 according to a third embodiment herein. The hinge configuration shown in FIG. 3 has two pairs of first hinge components 5 evenly spaced apart from each other at one end of multi-hinged longitudinal member 1 on main body 3. The spacing of the pairs of hinge components 5 in FIG. 3 are further apart compared with the spacing of the pairs of hinge components 5 shown in FIG. 2.

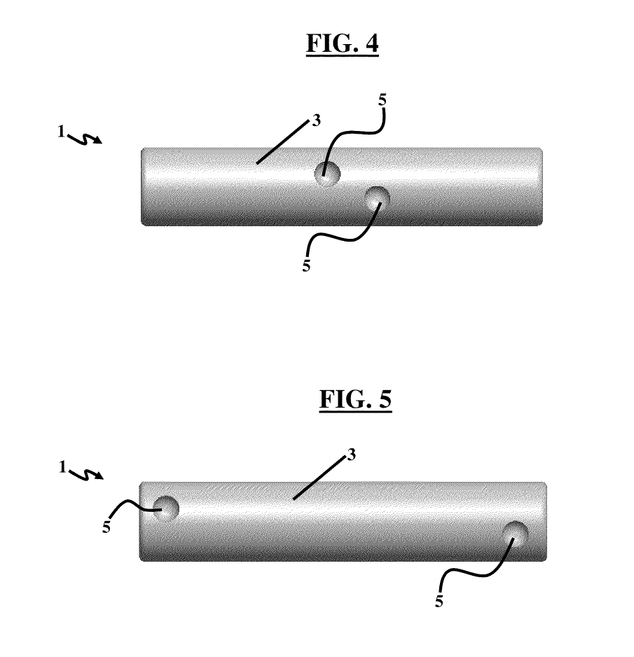

FIG. 4, with reference to FIGS. 1(A) through 3, illustrates a schematic diagram of a configuration of hinge components 5 on a multi-hinged longitudinal member 1 according to a fourth embodiment herein. The hinge configuration shown in FIG. 4 has two pairs of first hinge components 5 closes clustered in the middle of multi-hinged longitudinal member 1 on main body 3. FIG. 5, with reference to FIGS. 1(A) through 4, illustrates a schematic diagram of a configuration of hinge components 5 on a multi-hinged longitudinal member 1 according to a fifth embodiment herein. The hinge configuration shown in FIG. 5 has two pairs of first hinge components 5 evenly spaced apart from each other at opposite ends of multi-hinged longitudinal member 1 on main body 3.

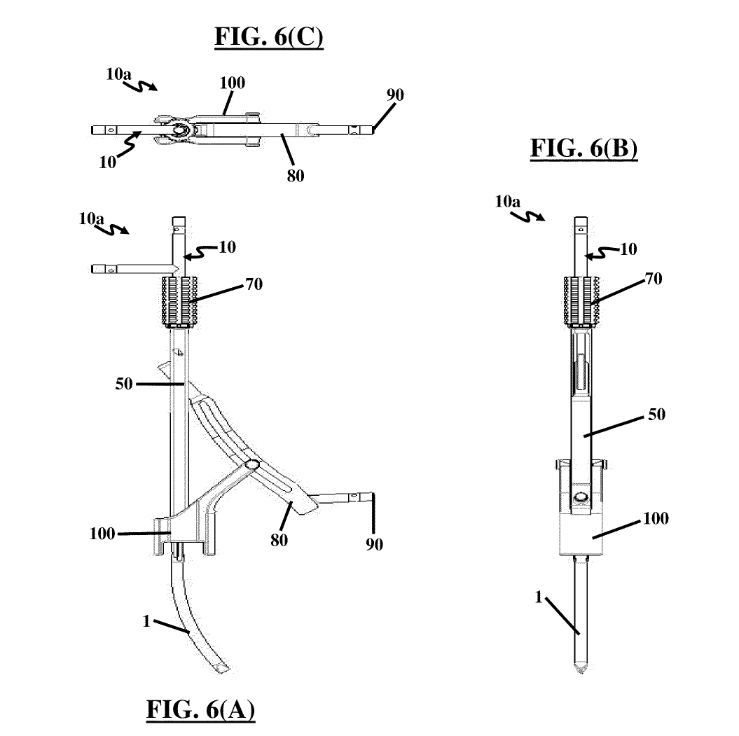

FIGS. 6(A) through 6(C), with reference to FIGS. 1(A) through 5, illustrate a schematic diagram of an insertion device 10a with an unsecured multi-hinged longitudinal member 1 according to an embodiment herein. As shown, left gripping arms 12 of left inner shaft 10 and right gripping arms 32 of right inner shaft 30 together grip multi-hinged longitudinal member 1. As described in more detail below, gripping arms 12, 32 may be forked-shaped, so that each will bend outward when gripping multi-hinged longitudinal member 1. In the configuration shown in FIGS. 6(A) through 6(C), multi-hinged longitudinal member 1 is not locked within gripping arms 12, 32 and easy removal of multi-hinged longitudinal member 1 from gripping arm 12, 32 is possible. In addition, outer sleeve 50 is shown in FIGS. 3(A) and 3(B) in a retracted position. In the retracted position, outer sleeve 50 is not in contact with gripping arms 12, 32. As discussed in further detail below, retraction of outer sleeve 50 is controlled by rotating knob 70.

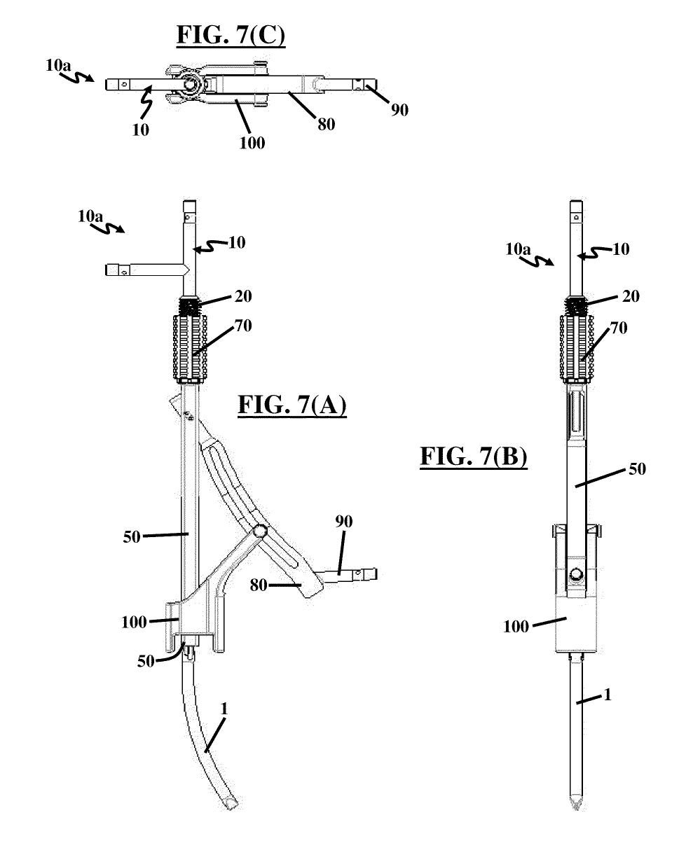

FIGS. 7(A) through 7(C), with reference to FIGS. 1(A) through 6(C), illustrate a schematic diagram of an insertion device 10a with a secured multi-hinged longitudinal member 1 according to an embodiment herein. Similar to FIGS. 6(A) through 6(C), gripping arms 12, 32 together grip multi-hinged longitudinal member 1. In FIGS. 7(A) through 7(C), however, multi-hinged longitudinal member 1 is locked and securely gripped by gripping arms 12, 32 to prevent easy removal of multi-hinged longitudinal member 1. As shown in FIG. 7(A), outer sleeve 50 is in an extended position. In the extended position, outer sleeve 50 is in contact with gripping arms 12, 32 to thereby compress (or pinch) gripping arms 12, 32 together and lock multi-hinged longitudinal member 1. As discussed in further detail below, extension of outer sleeve 50 is controlled by rotating knob 70.

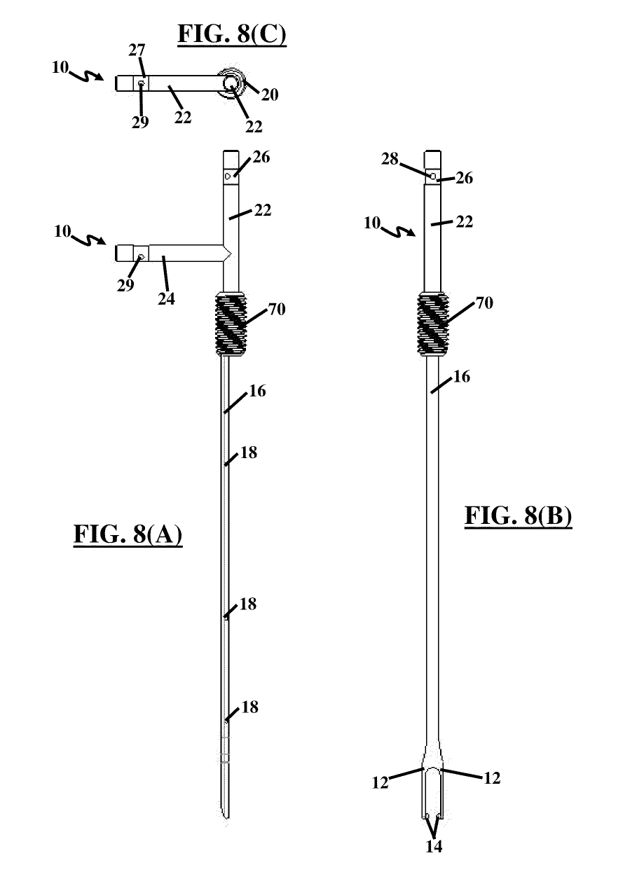

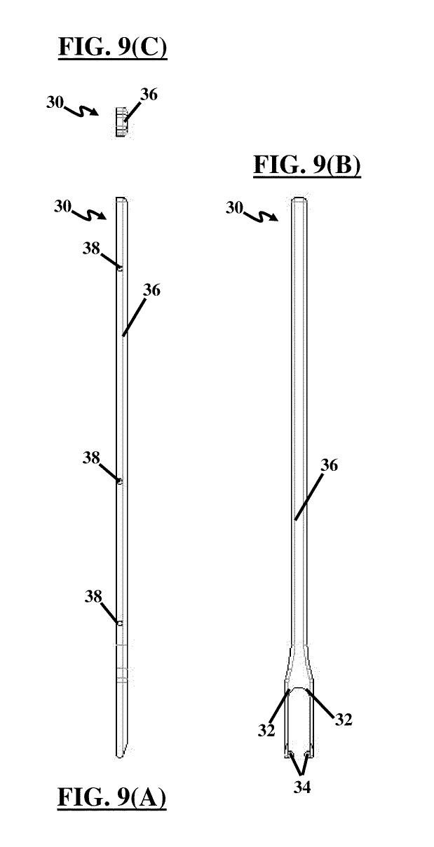

FIGS. 8(A) through 8(C), with reference to FIGS. 1(A) through 7(C) and 9(A) through 9(C), illustrate a schematic diagram of a left inner shaft 10 of the insertion device 10a according to an embodiment herein. As shown, left inner shaft 10 includes a pair of gripping arms 12 (or primary control arms), a pair of gripping nubs 14 (or second hinge components), a flat body 16, a plurality of pinholes 18, shaft threading 20 and a cylindrical body 22. In FIG. 8(B), gripping arms 12 are shown to be forked-shaped; however, other configurations are possible. The configuration of gripping arms 12 shown in FIG. 8(B) provides gripping arms 12 with some flex, so that each gripping arm 12 may bend slightly outwards to accommodate a multi-hinged longitudinal member 1 between gripping arms 12. The slight flex of gripping arms 12 also allows outer sleeve 50 to slide over gripping arms 12 and by so doing, compress (or pinch) gripping arms 12 together. When multi-hinged longitudinal member 1 is between the compressed gripping arms 12, multi-hinged longitudinal member 1 is effectively locked between gripping arms 12. In addition, as discussed in further detail below, multi-hinged longitudinal member 1 includes first hinge components 5 (shown in FIGS. 1(A) through 5) configured to be mated with gripping nubs 14 (as well as gripping nubs 34, shown in FIG. 9(B)).

As shown in FIG. 8(A), flat body 16 has a roughly rectangular shape, although other configurations are possible. Flat body 16 is the approximately the same size and shape as right inner shaft 30, shown in FIGS. 9(A) through 9(C) to allow flat body 16 and right inner shaft 30 to be joined by a plurality of connecting hinges 110. A plurality of pinholes 18 are bored into flat body 16 and slightly recessed to accommodate connecting hinges 110. As shown in FIGS. 8(A) and 8(B), one end of flat body 16 is securely coupled to shaft threading 20. Shaft threading 20 is configured to mate with rotating knob 70. In addition to being coupled to flat body 16, shaft threading is also coupled to cylindrical body 22. Cylindrical body 22 may be configured to engage a corresponding handle of a handle-like device (not shown). The cylindrical body 22 comprises a recessed collar 26 and hole 28 that may lock into a corresponding pin (not shown) of the handle-like device (not shown). The shaft 10 may further comprise a perpendicular body 24 configured transverse to the cylindrical body 22, wherein the perpendicular body 24 is also configured to engage another corresponding handle of a handle-like device (not shown). The perpendicular body 24 comprises a recessed collar 27 and hole 29 that may lock into a corresponding pin (not shown) of a handle-like device (not shown).

FIGS. 9(A) through 9(C), with reference to FIGS. 1(A) through 8(C), illustrate a schematic diagram of a right inner shaft 30 of the insertion device 10a according to an embodiment herein. As shown, right inner shaft 30 includes a pair of gripping arms 32 (or auxiliary control arms), a pair of gripping nubs 34 (or second hinge components), a flat body 36 and a plurality of pinholes 38. In FIG. 9(B), gripping arms 32 are shown to be forked-shaped; however, other configurations are possible. The configuration of gripping arms 32 shown in FIG. 9(B) provides gripping arms 32 with some flex, so that each gripping arm 32 may bend slightly outwards to accommodate a multi-hinged longitudinal member 1 between gripping arms 32. The slight flex of gripping arms 32 also allows outer sleeve 50 to slide over gripping arms 32, and by so doing, compress (or pinch) gripping arms 32 together. When multi-hinged longitudinal member 1 is between the compressed gripping arms 32, multi-hinged longitudinal member 1 is effectively locked between gripping arms 32. In addition, as discussed in further detail below, multi-hinged longitudinal member 1 includes a plurality of first hinge components 5 pairs (shown in FIGS. 1(A) through 5) configured to be mated with gripping nubs 34 (as well as gripping nubs 14, shown in FIG. 8(B)).

Also shown in FIG. 9(C), flat body 36 has a roughly rectangular shaped configuration, although other configurations are possible. Flat body 36 is approximately the same size and shape as flat body 16 of left inner shaft 10, shown in FIGS. 8(A) through 8(C) to allow flat body 36 and flat body 16 to be joined by a plurality of connecting hinges 110. In addition, a plurality of pinholes 18 are bored into flat body 36 and slightly recessed to accommodate connecting hinges 110.

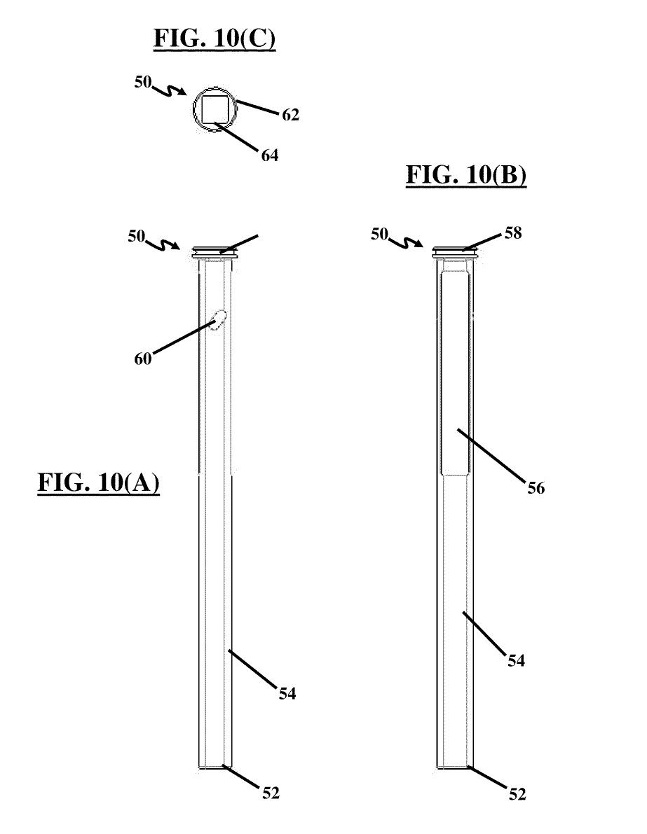

FIGS. 10(A) through 10(C), with reference to FIGS. 1(A) through 9(C) illustrate a schematic diagram of an outer sleeve 50 of the insertion device 10a according to an embodiment herein. As shown, outer sleeve 50 includes a distal end 52, a main body 54, an articulator cavity 56, a proximal end 58, access hole 60, a circular outer surface 62, and a polygonal inner surface 64. Distal end 52, as shown in FIG. 10(A), is the portion of outer sleeve 50 that covers gripping arms 12, 32 to compress gripping arms 12, 32 together and lock multi-hinged longitudinal member 1 between the compressed gripping arms 12, 32. Main body 54 is generally smooth and cylindrical in shape. Articulator cavity 56 is cut into main body 54 to accommodate the articulated movement of articulator 80. As described in further detail below, proximal end 58 attaches to rotating knob 70 to allow lateral translation of outer sleeve 50 with respect to inner shaft 10, 30. Access hole 60 is provided to ease assembly of inner shaft 10, 30 and insertion device 10a in general.

In addition, FIG. 10(C) shows outer sleeve having an outer circular surface 62, and an inner polygonal surface 64. Outer circular surface 62 is so configured to allow easy lateral movement within a percutaneous tube (not shown). Inner polygonal surface 64 is so configured to accommodate the rectangular cross-section of inner shaft 10, 30 and to prevent rotational movement of inner shaft 10 with respect to outer sleeve 50.

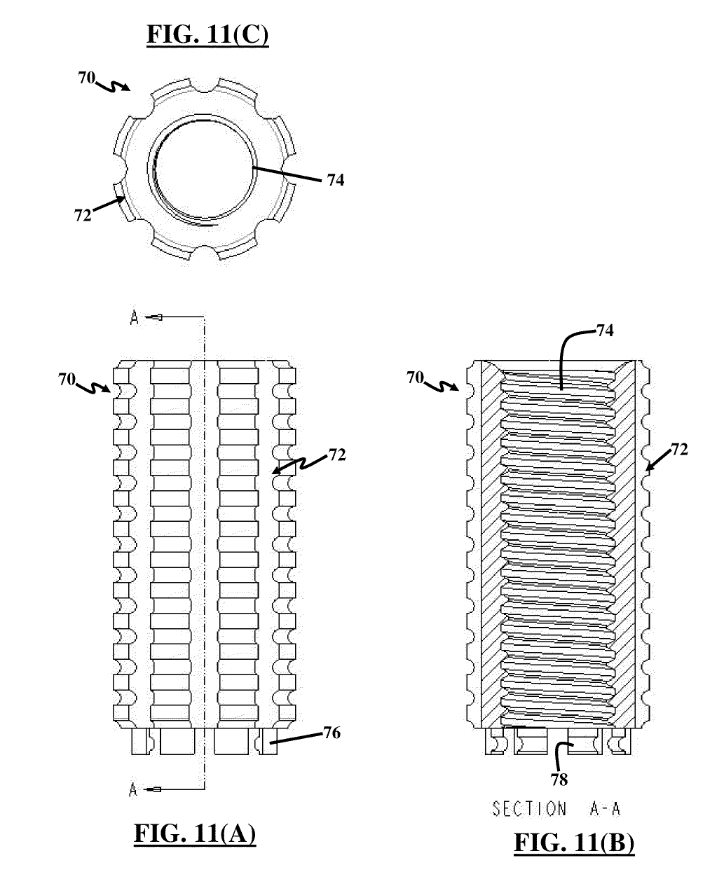

FIGS. 11(A) through 11(C), with reference to FIGS. 1(A) through 10(C), illustrate a schematic diagram of a rotating knob 70 of the insertion device 10a according to an embodiment herein. As shown, FIG. 11(B) is a cross-section taken along the A-A axis shown in FIG. 11(A). As shown in the various views, rotating knob 70 includes a textured exterior 72, a threaded interior 74, a connecting lip 76, and a connecting groove 78. Although rotating knob 70 is shown in FIGS. 11(A) and 11(B) as roughly cylindrically shaped, it is not limited to such a configuration. In addition, while textured exterior 72 is shown in FIG. 11(A) as a pattern of deep longitudinal grooves intersecting shallow lateral grooves, textured exterior 72 is not limited to the texture shown in FIG. 11(A). Rotating knob 70 also includes threaded interior 74, where the threads etched therein are configured to mate with complementary threads embedded on shaft threading 20, shown in FIGS. 8(A) and 8(B). In addition, outer sleeve 50 is configured to couple to rotating knob 70 by securely clipping onto connecting lip 76 and is held in place by connecting groove 78. When outer sleeve 50 is coupled to rotating knob 70, both outer sleeve 50 and rotating knob 70 move in unison. Consequently, when rotating knob 70 is mated with shaft threading 20 and a torque is applied to rotating knob 70, both rotating knob 70 and outer sleeve 50 are subjected to a linear translation as the threading of threaded interior 74 moves along the threading of shaft threading 20.

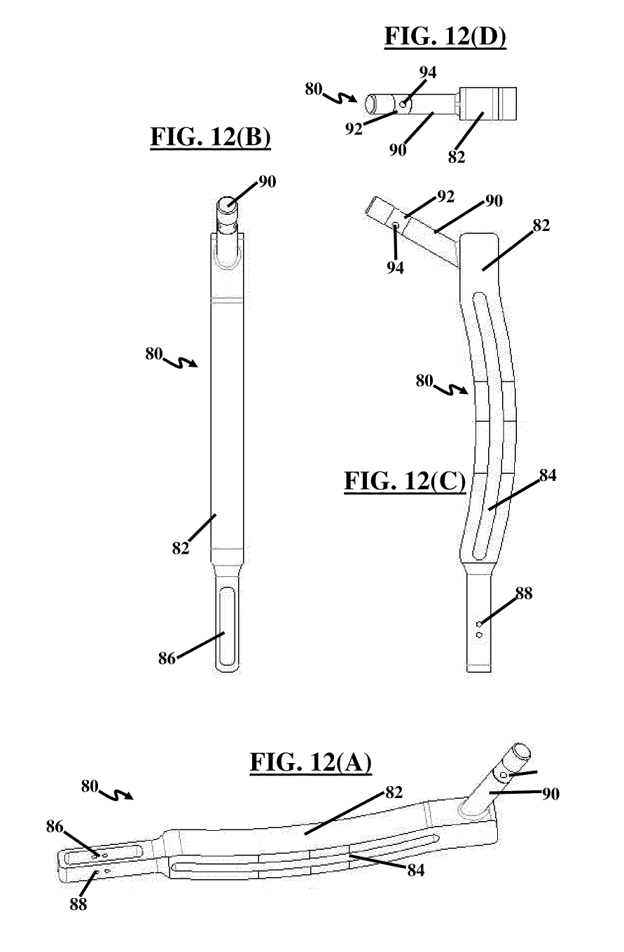

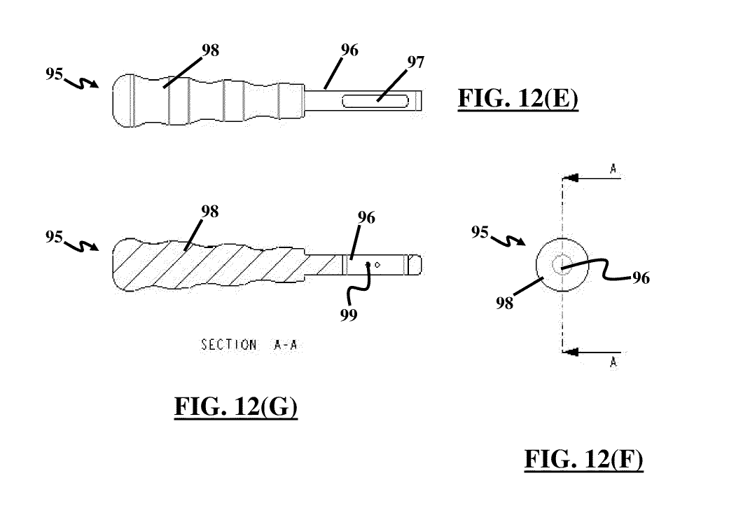

FIGS. 12(A) through 12(D), with reference to FIGS. 1(A) through 11(C), illustrate a schematic diagram of an actuator 80 of the insertion device 10a according to an embodiment herein. Actuator 80 provides depth control when inserting multi-hinged longitudinal member 1 during a minimally invasive surgical procedure. As shown in the various views of FIGS. 12(A) through 12(D), actuator 80 includes main body 82, translation cavity 84, inner shaft cavity 86, pinholes 88, control arm 90, control arm collar 92 and collar pinhole 94. As shown in FIG. 6(A), articulator 80 is coupled to base connector 100 by translation cavity 82. Additionally, articulator 80 is coupled to inner shaft 10, 30 by shaft cavity 86 and secured to inner shaft 10, 30 by pins inserted through pinholes 88. Although not shown in FIGS. 12(A) through 12(D), control arm 90 is provides leverage to allows sensitive depth adjustment and greater depth control while inserting multi-hinged longitudinal member 1 during a minimally invasive surgical procedure. The control arm 90 comprises a recessed collar 92 and hole 94 that may lock into a corresponding pin (not shown) of a handle-like device (not shown).

FIGS. 12(E) through 12(G), with reference to FIGS. 1(A) through 12(D), illustrate a schematic diagram of actuator 95 of the adjustable rod inserter 1 according to an embodiment herein. FIG. 12(G) is a cross-section of FIG. 16(F), cut along the A-A axis shown in FIG. 12(F). Similar to actuator 80, actuator 95 provides depth control when inserting longitudinal member 130 during a minimally invasive surgical procedure. As shown in the various views of FIGS. 12(E) through 12(G), actuator 95 includes main body 96, shaft cavity 97, control handle 98 and pinholes 99. While not shown, actuator 95 is coupled to inner shaft 10, 30 by shaft cavity 97 and secured to inner shaft 10, 30 by indent pins inserted through pinholes 99. Control handle 98 provides leverage to allow sensitive depth adjustment and greater depth control while inserting longitudinal member 130 during a minimally invasive surgical procedure. The control handle 98 is configured as a handle-like device to provide additional control and comfort during surgery.

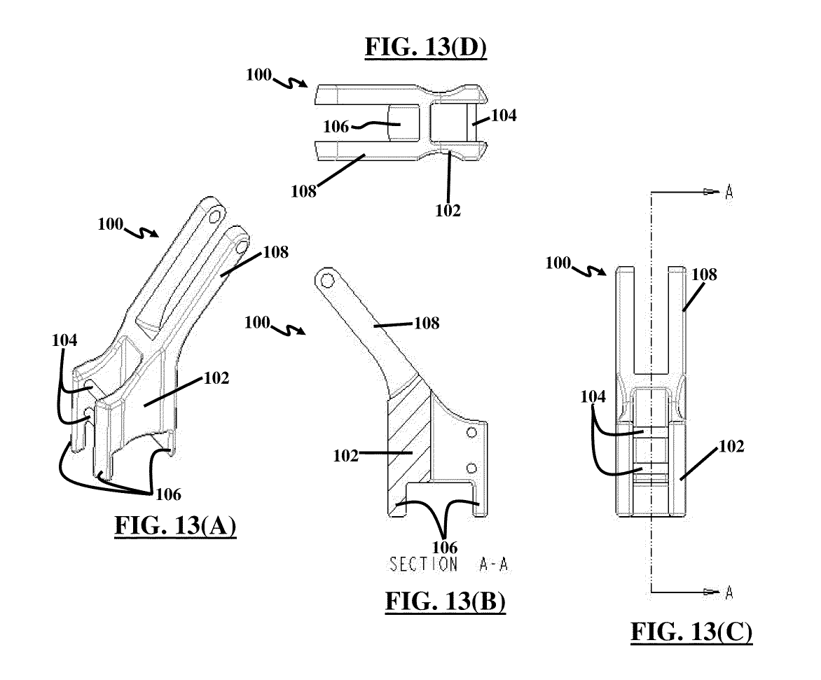

FIGS. 13(A) through 13(D), with reference to FIGS. 1(A) through 12(G), illustrate a schematic diagram of a base connector 100 of the insertion device 10a according to an embodiment herein. FIG. 13(B) is shown as a cross-section view cut along the A-A axis of FIG. 13(C). As shown, base connector 100 includes a main body 102, a plurality of support rails 104, a plurality of support legs 106, and a pair of connecting arms 108. As shown in FIG. 6(A), outer sleeve 50 is inserted through main body 102 and held in place by support rails 104. In addition, base connector 100 is anchored to a percutaneous tube (not shown) by support legs 106 and coupled to actuator 80 (via translation cavity 82, shown in FIGS. 12(A) through 12(C)) by connecting arms 108.

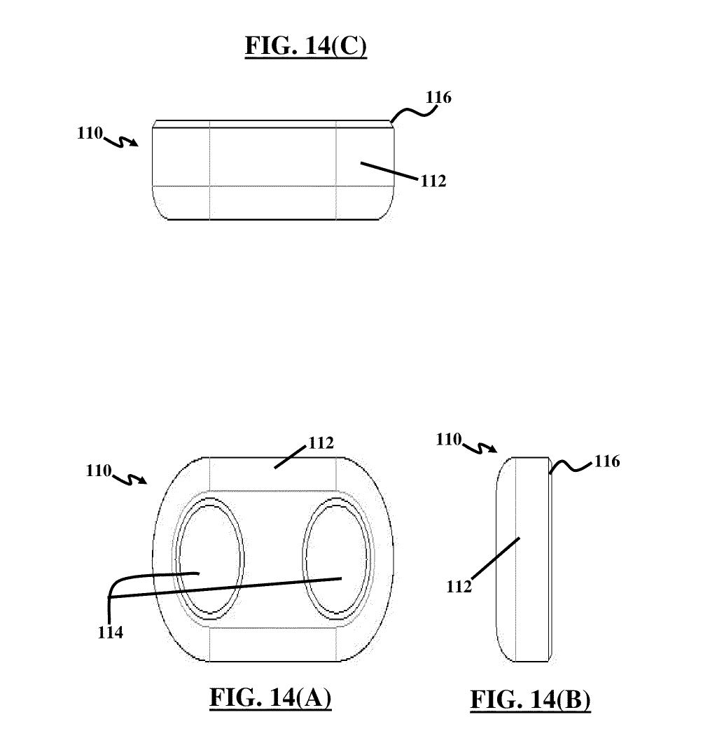

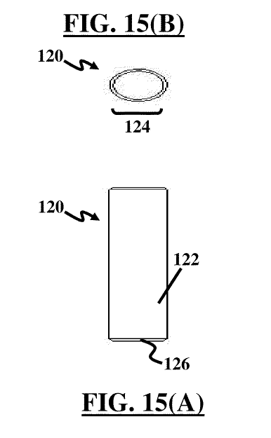

FIGS. 14(A) through 14(C), with reference to FIGS. 1(A) through 13(D), illustrate schematic diagram of a connecting hinge 110 of the insertion device 10a according to an embodiment herein. Connecting hinge 110 includes a main body 112 and a plurality of pinholes 114. In addition, connecting hinge 110 may further include a chamfered edge 126. FIG. 14(A) shows main body 112 with two pinholes 114 bored therethrough, and each pinhole 114 is dimensioned to securely mate with a pin 120. As discussed previously, connecting hinge 110 securely couples left inner shaft 10 to right inner shaft 30 by aligning pinholes 114 with pinholes 18, 38 and securing a pin 120 through the aligned pinholes. Furthermore, pinholes 18, 38 are sufficiently recessed into inner shaft 10, 30 to permit connecting hinge 110 to sit flush with the outer surface of inner shaft 10, 30 and allow linear translation of inner shaft 10, 30 within outer sleeve 50.

FIGS. 15(A) through 15(B), with reference to FIGS. 1(A) through 14(C), illustrate a schematic diagram of a pin 120 of the insertion device 10a according to an embodiment herein. As shown, pin 120 includes a main body 122, which has a diameter 124. In addition, pin 120 may further include chamfered edges 126. Furthermore, diameter 124 is sufficient to securely mate with pinholes 18, 38 and pinholes 114.

FIG. 16, with reference to FIGS. 1(A) through 15(B), illustrates a schematic diagram of the tip end of insertion device 130 which may be used to maneuver the longitudinal member 1 according to an embodiment herein. Insertion device 130 is configured as a scissor-action tool with a clamp-like mechanism that generally includes a tip end 132 and a distally located handle end (not shown). Insertion device 130 further includes two gripping arms 134, 136 connected together by a pivot mechanism 137 such as a screw or pin to provide rotation means for the gripping arms 134, 136 to move in relation to one another. Furthermore, while not shown, the handle end of insertion device 130 may include a locking mechanism operatively connected to the gripping arms 134, 136. Insertion device 130 opens/closes in a scissor-like fashion, such that when the gripping arms 134, 136 close they create a generally square-shaped notch 138 that may accommodate the multi-hinged longitudinal member 1. The notch 138 can grip the first hinge components 5 of the longitudinal member 1 especially when the hinge components 5 are embodied as projections outwardly protruding from the longitudinal member 1.

FIGS. 17(A) and 17(B), with reference to FIGS. 1(A) through 16, illustrate schematic diagrams of an alternate configuration of a tip end 142 of an insertion device 139 according to an embodiment herein, wherein only one of the tip ends 142 is illustrated in the drawings. Insertion device 136 is also configured as a scissor-action tool, such that when two complementary arms 140 are squeezed together, they similarly form a substantially square-shaped hole through the joining of notch 152 from one tip end 142 with the corresponding notch 152 from another tip end 142. Each tip end 142 comprises a prong tip 144 positioned on a base 146 to allow for gripping of multi-hinged longitudinal member 1, whereby the gripping prong tip 144 is dimensioned and configured to engage first hinge components 5 of multi-hinged longitudinal member 1. In this regard, tip 144 can grip the hinge components 5 of the longitudinal member 1 especially when the first hinge components 5 are embodied as dimples indented in the longitudinal member 1. The prong tip 144 is dimensioned and configured to have a sloping wall 148, which generally aligns with a sloping surface of the base 146. A corresponding sloping wall 150 of the gripping arm 140 forms the notch 152 in each gripping arm 140.

According to the embodiments herein, each of nubs 14, 34, notch 138, and tip 144 acts as a second hinge component that is complementary to the first hinge components 5 of the longitudinal member 1. In other words, the first hinge components 5 and the second hinge component 14, 34, 138, 144 together form a hinge-like connection to allow the longitudinal member 1 to rotate, articulate, or otherwise move.

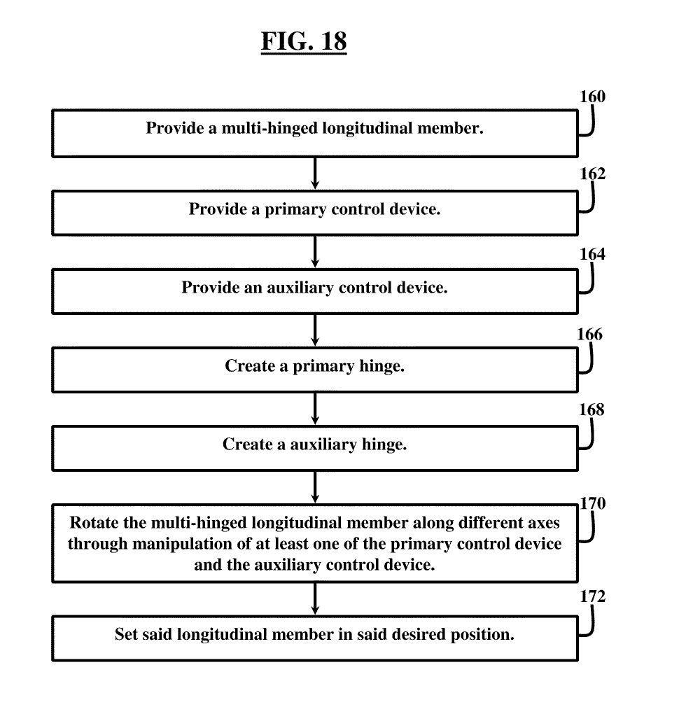

FIG. 18, with reference to FIGS. 1(A) through 17(B), illustrates a flow diagram of using a multi-hinged longitudinal member 1 according to an embodiment herein. In step 160 of the method shown in FIG. 18, a multi-hinged longitudinal member (e.g., multi-hinged longitudinal member 1) is provided. In step 162, a primary control device (e.g., left inner shaft 10 or gripping arm 134, 136, 139) is provided. In step 164, an auxiliary control device (e.g., right inner shaft 30, or gripping arm 134, 136, 139) is provided. In step 166, a primary hinge is created (e.g., by coupling first hinge components 5 with gripping nubs 14, or tip 144 or coupling first hinge components 5 with second hinge components 138). In step 168, an auxiliary hinge is created (e.g., by coupling first hinge components 5 with gripping nubs 34, or coupling first hinge components 5 with second hinge components 138). Step 170 describes rotating the multi-hinged longitudinal member along different axes through manipulation of at least one of the primary control device and the auxiliary control device. In step 172, the method of FIG. 18 describes setting said longitudinal member in the desired position.

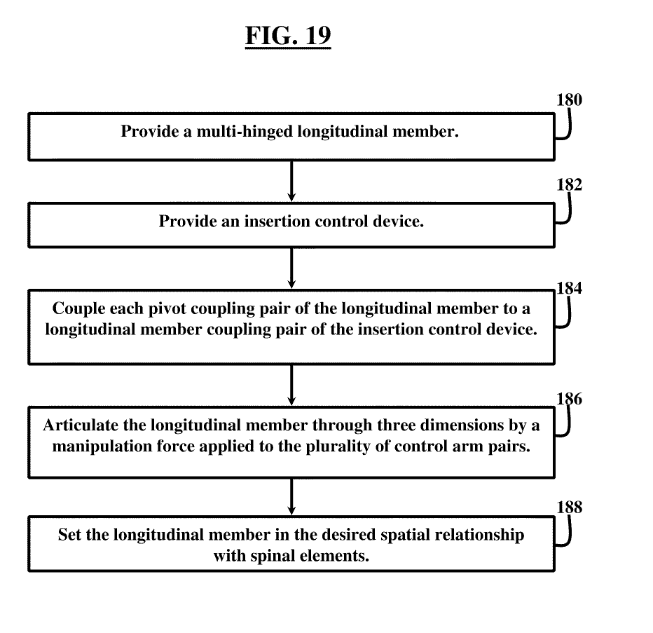

FIG. 19, with reference to FIGS. 1(A) through 18, illustrates a flow diagram of another use of the multi-hinge longitudinal member according to an embodiment herein. In step 180 of the method shown in FIG. 19, a multi-hinged longitudinal member (e.g., multi-hinged longitudinal member 1) is provided. In step 182, an insertion control device (e.g., insertion device 10 or insertion device 130, 139) is provided. Step 184 describes coupling each pivot coupling pair of the longitudinal member 1 to a longitudinal member coupling pair of the insertion control device (e.g., by coupling first hinge components 5 with gripping nubs 14, or tip 144 or coupling first hinge components 5 with second hinge components 138, by coupling first hinge components 5 with gripping nubs 34, or coupling first hinge components 5 with second hinge components 138). Step 186 describes articulating the longitudinal member 1 through three dimensions by a manipulation force applied to the plurality of control arm pairs. In step 188, the method of FIG. 19 describes setting the longitudinal member 1 in the desired spatial relationship with spinal elements.

Generally, the embodiments herein provide a longitudinal member 1 comprising multiple hinge components 5, which may be embodied as dimples indented into the longitudinal member 1 or as projections outwardly protruding from the longitudinal member 1. An inserter device 10a, 130, 139 comprises corresponding means for actuating the hinge components 5, wherein the actuation means may be embodied as nubs 14, 34, notch 138, or tip 144. In one embodiment, one inserter device engages the multiple hinge components 5. In another embodiment, multiple inserter devices are used together to engage the multiple hinge components 5. The actuation of the multiple hinge components 5 allows for the rotation of the longitudinal member 1 along different axes of rotation, to permit enhanced manipulation of the longitudinal member 1, and for ease of rotation of the longitudinal member 1.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A method of setting a multi-hinged longitudinal member in a desired position during a spinal surgery, said method comprising:
providing said multi-hinged longitudinal member, wherein said multi-hinged longitudinal member comprises:
a primary first hinge component pair; and
an auxiliary first hinge component pair;
providing a primary control device, wherein said primary control arm comprises:
a primary control arm pair; and
a primary second hinge component pair,
wherein each primary control arm of said primary control arm pair is coupled to a primary second hinge component of said primary second hinge component pair;
providing an auxiliary control device, wherein said auxiliary control arm comprises:
an auxiliary second hinge component pair; and
an auxiliary control arm pair,
wherein each auxiliary control arm of said auxiliary control arm pair is coupled to an auxiliary second hinge component of said auxiliary second hinge component pair;
creating a primary hinge by connecting each primary first hinge component to a unique primary second hinge component to create a primary hinge;
creating a auxiliary hinge by connecting each said auxiliary first hinge component to a unique auxiliary second hinge component to create an auxiliary hinge;
rotating said multi-hinged longitudinal member along different axes through manipulation of at least one of said primary control device and said auxiliary control device; and
setting said multi-hinged longitudinal member in said desired position.

2. The method of claim 1, further comprising:
setting a center hinge on said multi-hinged longitudinal member;
holding said primary hinge stationary; and
articulating said auxiliary hinge.

3. The method of claim 1, further comprising:
rotating said multi-hinged longitudinal member along a lateral axis of said multi-hinged longitudinal member;
articulating said primary hinge in a first direction; and
articulating said auxiliary hinge in a second direction,
wherein said first direction is a complement of said second direction.

4. The method of claim 1, further comprising:
rotating said multi-hinged longitudinal member along a longitudinal axis of said multi-hinged longitudinal member;
articulating said primary hinge in a first direction; and
articulating said auxiliary hinge in said first direction.

5. A method of setting a multi-hinged longitudinal member in a desired spatial relationship with spinal elements during minimally invasive spinal surgery, said method comprising:
providing a longitudinal member comprising multiple hinge components;
providing an inserter device comprising means for actuating said multiple hinge components; and
actuating said multiple hinge components of said longitudinal member.

6. The method of claim 5, wherein the actuation of said multiple hinge components allows for rotation of said longitudinal member along different axes of rotation.

7. The method of claim 5, wherein said longitudinal member comprises elongated side surfaces, wherein said multiple hinge components are configured along said side surfaces.

8. The method of claim 5, wherein said longitudinal member comprises a cylindrical body.

9. The method of claim 5, wherein said multiple hinge components comprise dimples indented into said longitudinal member.

10. The method of claim 5, wherein said multiple hinge components comprise projections outwardly protruding from said longitudinal member.

11. The method of claim 9, wherein said means for actuating said multiple hinge components comprises multiple projections that insert into said dimples.

12. The method of claim 10, wherein said means for actuating said multiple hinge components comprises multiple notches that engage said projections.

13. The method of claim 5, further comprising providing multiple inserter devices comprising means for actuating said multiple hinge components.

14. An apparatus for setting a multi-hinged longitudinal member in a desired spatial relationship with spinal elements during minimally invasive spinal surgery, said apparatus comprising:
a longitudinal member comprising multiple hinge components; and
at least one inserter device comprising means for actuating said multiple hinge components,
wherein the actuation of said multiple hinge components allows for rotation of said longitudinal member along different axes of rotation.

15. The apparatus of claim 14, wherein said longitudinal member comprises elongated side surfaces, wherein said multiple hinge components are configured along said side surfaces.

16. The apparatus of claim 14, wherein said longitudinal member comprises a cylindrical body.

17. The apparatus of claim 14, wherein said multiple hinge components comprise dimples indented into said longitudinal member.

18. The apparatus of claim 14, wherein said multiple hinge components comprise projections outwardly protruding from said longitudinal member.

19. The apparatus of claim 17, wherein said means for actuating said multiple hinge components comprise multiple projections that insert into said dimples.

20. The apparatus of claim 18, wherein said means for actuating said multiple hinge components comprise notches that engage said projections.
